(19) United States
(12) Patent Application Publication
(10) Pub. No.: US 2025/0049757 A1
(43) Pub. Date: Feb. 13, 2025

(54) COMBINATORY THERAPY FOR CANCER USING BIPHENYL COMPOUND AND IMMUNE CHECKPOINT MOLECULE REGULATOR

(71) Applicants: Taiho Pharmaceutical Co. Ltd., Tokyo (JP); Astex Pharmaceuticals, Inc., Pleasanton, CA (US)

(72) Inventors: Akiko Osada, Ibaraki (JP); Toshihiro Shibutani, Ibaraki (JP)

(21) Appl. No.: 18/697,416

(22) PCT Filed: Sep. 29, 2022
(86) PCT No.: PCT/JP2022/036343

§ 371 Date: Mar. 29, 2024

(30) Foreign Application Priority Data
Sep. 30, 2021 (JP) 2021-161733

(51) Int. Cl. A61K 31/40; A61K 39/00; A61P 35/00; C07K 16/28

(52) U.S. Cl. CPC A61K 31/40; A61P 35/00; C07K 16/2818; A61K 2039/505

(57) ABSTRACT

Provided is a novel cancer treatment method which exhibits a remarkably excellent antitumor effect with little side effects. An antitumor agent includes a biphenyl compound for use in a treatment of a subject having cancer. The treatment comprises administering an immune checkpoint molecule regulator to the subject.

### TECHNICAL FIELD

The present disclosure relates to an antitumor agent comprising a biphenyl compound or a salt thereof in combination with an immune checkpoint molecule regulator; and an antitumor effect enhancing agent and a kit preparation.

### BACKGROUND ART

Lysine-specific demethylase 1 (LSD1) is an epigenetic enzyme that mono- or dimethylates histone H3 lysine 4 (H3K4) and H3 lysine 9 (H3K9). In addition to its function of epigenetic regulator, LSD1 regulates some non-histone substrates including DNMT1, p53, STAT3, and E2F1 (Non-Patent Literature 1). LSD1 is also a component of different multi-protein complexes and its association with various gene regulatory proteins has been demonstrated (Non-Patent Literature 2). A large number of studies have highlighted the pivotal role of LSD1 in several cellular processes in normal and cancer cells such as control of stemness, differentiation, cell motility, epithelial-to-mesenchymal transition (Non-Patent Literature 3).

LSD1 is highly expressed and is associated with poor prognosis in many cancers (Non-Patent Literature 4). It is involved in various stages of cancer, including development, progression, metastasis, and recurrence after therapy. Targeting LSD1 has been recognized as a promising strategy for cancer treatment in recent years. Several LSD1 inhibitors are undergoing clinical assessment for cancer therapy (Non-Patent Literature 1).

On the other hand, the immunological system is an important mechanism for self-defense against various diseases caused by in vivo and in vitro factors. Deterioration of the functions of the immunological system has pathologically adverse effects such as development of infectious diseases by bacteria and viruses, development of tumors, and delay of recovery from injuries and diseases. Therefore, activation of the immunological system is very important for prevention and treatment of various diseases. As one of new methods for treating cancer, cancer immunotherapy is being developed.

Activation of adaptive immunological reaction is initiated by binding of an antigenic peptide-MHC complex to a T-cell receptor (TCR). Further, this binding is regulated by costimulation or coinhibition due to binding between the B7 family which is a costimulatory molecule and the CD28 family which is a receptor of the B7 family. That is, for T-cells to be activated in an antigen-specific manner, two characteristic signal transduction events are required, and T-cells which have not undergone costimulation from the B7 family and have undergone only antigen-stimulation are turned into a nonresponsive state (anergy), so that immunological tolerance is induced in the T-cells.

By taking advantage of this mechanism, cancer cells suppress activation of antigen-specific T-cells to escape from the immune surveillance, and continuously grow. Thus, for cancer treatment, it is considered effective that by enhancement of costimulation and blocking of coinhibition, an in vivo antitumor immune response is induced in a cancer patient to control immune escape of a tumor, and various cancer immunotherapies targeted at costimulatory molecules (stimulatory costimulatory molecules) or coinhibitory molecules (inhibitory costimulatory molecules) have been proposed (Patent Literature 6). For example, as an immune checkpoint molecule regulator for activating T-cells by inhibiting binding of PD-1 and ligands thereof (PD-L1 and PD-L2), nivolumab (human-type IgG4 monoclonal antibody against human PD-1) is used for malignant melanoma etc. (Patent Literature 1 and Non Patent Literature 5).

A biphenyl compound 4-[5-[(3S)-3-aminopyrrolidine-1-carbonyl]-2-[2-fluoro-4-(2-hydroxy-2-methyl-propyl)phenyl]phenyl]-2-fluoro-benzonitrile, referred to as a compound represented by the formula (I) below, or a salt thereof is known as a LSD1 inhibitor (Patent Literature 2), and a combination of the LSD1 inhibitor and another antitumor agent has been heretofore reported (Patent Literature 3).

However, the biphenyl compound represented by the formula (I) and an immune checkpoint molecule regulator have not been used in combination yet. In addition, the immunostimulatory action of the biphenyl compound represented by the formula (I) is not known.

### CITATION LIST

### Patent Literature

PTL 1: International Publication No. WO2004/004771
PTL 2: International Publication No. WO2017/090756
PTL 3: International Publication No. WO2018/216795

### Non Patent Literature

NPL 1: J. Hematol. Oncol, 12:129 (2019)
NPL 2: Cancers (Basel), 11:324 (2019)
NPL 3: Crit. Rev. Eukaryot. Gene Expr, 22:53-59 (2012)
NPL 4: Pharmacol. Res, 164:105335 (2020)
NPL 5: N. Engl. J. Med., 366:2443-2454 (2012)

### SUMMARY OF INVENTION

### Technical Problem

An object of the present disclosure is to provide a novel cancer treatment method which exhibits a remarkably excellent antitumor effect with little side effects.

### Solution to Problem

The combination of 4-[5-[(3S)-3-aminopyrrolidine-1-carbonyl]-2-[2-fluoro-4-(2-hydroxy-2-methyl-propyl)phenyl]phenyl]-2-fluoro-benzonitrile (“Compound A”) or a salt thereof and an immune checkpoint molecule regulator was studied, and the antitumor effect of the combination was investigated. As a result, it was found that the antitumor effect is more remarkably enhanced without causing severe side effects as compared to a case where a single agent is used.

Specifically, in one embodiment, the present disclosure provides the following aspects [1] to [26].

[1] An antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in a treatment of a subject having cancer, wherein the treatment comprises administering an immune checkpoint molecule regulator to the subject.

[2] The antitumor agent according to [1], wherein the immune checkpoint molecule regulator is at least one or more selected from the group consisting of a PD-1 pathway antagonist and a CTLA-4 pathway antagonist.

[3] The antitumor agent according to [1] or [2], wherein the immune checkpoint molecule regulator is a PD-1 pathway antagonist.

[4] The antitumor agent according to [2] or [3], wherein the PD-1 pathway antagonist is at least one or more selected from the group consisting of an anti-PD-1 antibody, an anti-PD-L1 antibody and an anti-PD-L2 antibody.

[5] The antitumor agent according to [2] or [3], wherein the PD-1 pathway antagonist is an anti-PD-1 antibody or an anti-PD-L1 antibody.

[6] The antitumor agent according to [2] or [3], wherein the PD-1 pathway antagonist is an anti-PD-1 antibody.

[7] The antitumor agent according to [2], wherein the CTLA-4 pathway antagonist is an anti-CTLA-4 antibody.

[8] An antitumor effect enhancing agent for an immune checkpoint molecule regulator, comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof as an active ingredient.

[9] An antitumor agent for treating a cancer patient given an immune checkpoint molecule regulator, the antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

[10] An antitumor agent for treating a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof, the antitumor agent comprising an immune checkpoint molecule regulator.

[11] 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in treating a tumor by administration thereof in combination with an immune checkpoint molecule regulator.

[12] 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in enhancing the antitumor effect of an immune checkpoint molecule regulator.

[13] 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in treating a tumor in a cancer patient given an immune checkpoint molecule regulator.

[14] An immune checkpoint molecule regulator for use in treating a tumor in a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

[15] A combination of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator for use in treating a tumor.

[16] Use of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for producing an antitumor agent which is administered in combination with an immune checkpoint molecule regulator.

[17] Use of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for producing an antitumor effect enhancing agent for enhancing the antitumor effect of an immune checkpoint molecule regulator.

[18] Use of 4-[5-[(3S)-3-aminopyrrolidine-1-carbonyl]-2-[2-fluoro-4-(2-hydroxy-2-methyl-propyl) phenyl]phenyl]-2-fluoro-benzonitrile or a salt thereof for producing an antitumor agent for treating a cancer patient given an immune checkpoint molecule regulator.

[19] Use of an immune checkpoint molecule regulator for producing an antitumor agent for treating a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

[20] Use of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator for producing an antitumor agent.

[21] A method for treating a tumor, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator in combination.

[22] A method for enhancing the antitumor effect of an immune checkpoint molecule regulator, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

[23] A method for treating a tumor in a cancer patient given an immune checkpoint molecule regulator, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

[24] A method for treating a tumor in a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof, comprising administering to a subject in need thereof an immune checkpoint molecule regulator.

[25] A method for treating a tumor, comprising administering to a subject in need thereof a combination of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator.

[26] A pharmaceutical composition for preventing and/or treating a tumor, comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator.

Further, another exemplary embodiment relates to:
An antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator which are administered in combination.
A method of treating a cancer patient showing higher level of gMDSC using antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and/or an immune checkpoint molecule regulator as monotherapy or combination therapy.
A method of treating a cancer, comprising predicting a chemotherapy using an antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and/or an immune checkpoint molecule regulator in a cancer patient based on the amount of granulocytic myeloid-derived suppressor cells (gMDSC) in a sample isolated from the cancer patient, and administering the antitumor agent to any cancer patient predicted to sufficiently respond to the chemotherapy using the antitumor agent.
A method of predicting a chemotherapy for cancer treatment, comprising predicting a chemotherapy using an antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and/or an immune checkpoint molecule regulator in a cancer patient based on the amount of granulocytic myeloid-derived suppressor cells (gMDSC) in a sample isolated from the cancer patient.

### Advantageous Effects of Invention

The antitumor agent of the present disclosure makes it possible to perform cancer treatment with high antitumor effects while suppressing development of side-effects.

### BRIEF DESCRIPTION OF DRAWINGS

FIG. 1A shows the ratio of granulocytic myeloid-derived suppressor cells (gMDSC, same as polymorphonuclear-MDSC) in tumor infiltrating leucocytes.

FIG. 1B shows the ratio of CD8 positive T cells in tumor infiltrating leucocytes.

FIG. 2A shows the anti-tumor effect of Compound A in a wildtype (immune proficient) mouse model which has been transplanted with a mouse colon cancer cell line MC38.

FIG. 2B shows the anti-tumor effect of Compound A in an immune deficient mouse model which has been transplanted with a mouse colon cancer cell line MC38.

FIG. 3 shows the effect of a combination of Compound A and an anti-mouse PD-1 antibody in a wildtype mouse model which has been transplanted with a mouse colon cancer cell line MC38.

### DESCRIPTION OF EMBODIMENTS

4-[5-[(3S)-3-aminopyrrolidine-1-carbonyl]-2-[2-fluoro-4-(2-hydroxy-2-methyl-propyl)phenyl]phenyl]-2-fluoro-benzonitrile is depicted in the following structure:

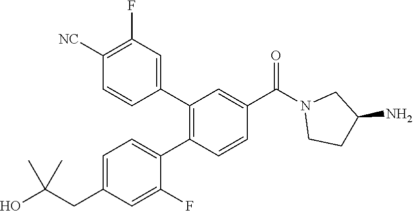 [SMILES: CC(C)(O)Cc1ccc(-c2ccc(C(=O)N3CC[C@H](N)C3)cc2-c2ccc(C#N)c(F)c2)c(F)c1]

In the present disclosure, the above compound is described as “Compound A”. Compound A is described as Example compound 37 of PCT Publication No. WO2017/090756, the disclosure of which is incorporated by reference herein in its entirety. Compound A can be produced by any known methods in the art, including, but not limited to, those methods described in PCT Publication No. WO2017/090756 and WO2021/095835, the disclosure of which is incorporated by reference herein in its entirety.

The present disclosure relates to an antitumor agent (which can be interchangeably used with “a medicament for treating tumor”), an antitumor effect enhancing agent, and a kit preparation, comprising Compound A or a salt thereof and an immune checkpoint molecule regulator (in particular, anti-PD-1 antibody), which are administered in combination; use thereof; a method for treating a tumor; and a method for enhancing an antitumor effect.

Compound A or a salt thereof used in the present disclosure may be in the form of crystals. Single crystals and polymorphic crystal mixtures are included within the scope of Compound A or a salt thereof. Such crystals can be produced by crystallization according to a crystallization method known in the art. Compound A or a salt thereof may be a solvate (e.g., a hydrate) or a non-solvate. Any of such forms are included within the scope of the compound of the present disclosure or a salt thereof.

Compounds A labeled with an isotope (e.g., ³H, ¹⁴C, ³⁵S, and ¹²⁵I) are also included within the scope of Compound A or a salt thereof used in the present disclosure.

The salts of Compound A used in the present disclosure refer to common salts used in the field of organic chemistry. Examples of such salts include base addition salts, and acid addition salts. The salts of Compound A are preferably pharmaceutically acceptable salts.

Examples of base addition salts include alkali metal salts, such as sodium salts and potassium salts; alkaline earth metal salts, such as calcium salts and magnesium salts; ammonium salts; and organic amine salts, such as trimethylamine salts, triethylamine salts, dicyclohexylamine salts, ethanolamine salts, diethanolamine salts, triethanolamine salts, procaine salts, and N,N′-dibenzylethylenediamine salts.

Examples of acid addition salts include inorganic acid salts, such as hydrochloride, sulfate, nitrate, phosphate, and perchlorate; organic acid salts, such as acetate, formate, maleate, fumarate, tartrate, citrate, ascorbate, benzoate and trifluoroacetate; and sulfonates such as methanesulfonate, isethionate, benzenesulfonate, and p-toluenesulfonate.

One example of a salt of Compound A is benzoic acid salt or benzoate salt. Another example of a salt of Compound A is sorbic acid salt, succinic acid salt, L-tartaric acid salt, or hydrochloric acid salt. Yet another example of a salt of Compound A is hemifumarate, mono-oxalate, mesylate, esylate, maleate, mono-fumarate, or hemi-oxalate.

The immune checkpoint molecule regulator in the present disclosure directly acts on immune checkpoint molecules, so that an in vivo antitumor immune response is induced in a cancer patient to control immune escape of a tumor.

Examples of the immune checkpoint molecule regulator include substances which promote the functions of costimulatory molecules (stimulatory costimulatory molecules), or substances which suppress the functions of coinhibitory molecules (inhibitory costimulatory molecules). Examples of the immune checkpoint molecules include B7 family (B7-1, B7-2, PD-L1, PD-L2, etc.), CD28 family (CTLA-4, PD-1, etc.), TNF superfamily (4-1BBL and OX40L), TNF receptor superfamily (4-1BB and OX40) molecules, TIGIT pathway molecules (TIGIT etc.) and LAG-3 pathway molecules (LAG-3 etc.). For the immune checkpoint molecule regulator, a substance targeted at one or more of the immune checkpoint molecules can be used. Examples of the substance include PD-1 pathway antagonists, ICOS pathway agonists, CTLA-4 pathway antagonists, CD28 pathway agonists, BTLA pathway antagonists, 4-1BB pathway agonists, TIGIT pathway antagonist and LAG-3 pathway antagonist.

In the present disclosure, the immune checkpoint molecule regulator is preferably at least one or more selected from the group consisting of a PD-1 pathway antagonist, an ICOS pathway agonist, a CTLA-4 pathway antagonist and a CD28 pathway agonist, more preferably at least one or more selected from the group consisting of a PD-1 pathway antagonist and a CTLA-4 pathway antagonist, still more preferably a PD-1 pathway antagonist from the viewpoint of suppressing side-effects.

The PD-1 pathway antagonist inhibits immunosuppressive signals from PD-1 expressed on T-cells. PD-L1 or PD-L2 is a ligand of PD-1, and examples of the PD-1 pathway antagonist include anti-PD-1 antibody, anti-PD-L1 antibody, anti-PD-L2 antibody, PD-1 extracellular domain, PD-L1 extracellular domain, PD-L2 extracellular domain, PD-1-Ig (fusion protein of PD-1 extracellular domain and FC region of immunoglobulin (Ig)), PD-L1-Ig, PD-L2-Ig, PD-1 siRNA, PD-L1 siRNA and PD-L2 siRNA. The PD-1 pathway antagonist is preferably an anti-PD-1 antibody, an anti-PD-L1 antibody or an anti-PD-L2 antibody, more preferably an anti-PD-1 antibody or an anti-PD-L1 antibody, particularly preferably an anti-PD-1 antibody.

The CTLA-4 pathway antagonist inhibits immunosuppressive signals from CTLA-4 expressed on T-cells. B7-1 (CD80) or B7-2 (CD86) is a ligand of CTLA-4. The CTLA-4 pathway antagonist is preferably an anti-CTLA-4 antibody, a CTLA-4 extracellular domain, CTLA-4-Ig, an anti-B7-1 (CD80) antibody or an anti-B7-2 (CD86) antibody, more preferably an anti-CTLA-4 antibody or CTLA-4-Ig, particularly preferably an anti-CTLA-4 antibody.

In one of the aspects of the present disclosure, the immune checkpoint molecule regulator is preferably at least one or more selected from the group consisting of an anti-PD-1 antibody, an anti-PD-L1 antibody and an anti-CTLA-4 antibody, more preferably at least one or more selected from the group consisting of an anti-PD-1 antibody and an anti-PD-L1 antibody, particularly preferably an anti-PD-1 antibody.

Examples of the antibodies include immunoglobulins (IgA, IgD, IgE, IgG, IgM, IgY, etc.), Fab fragments, F(ab′)₂ fragments, single-stranded antibody fragments (scFv), single domain antibodies, and Diabody (Nat. Rev. Immunol., 6:343-357, 2006). These antibodies include monoclonal antibodies or polyclonal antibodies such as human antibodies, humanized antibodies, chimeric antibodies, mouse antibodies, llama antibodies and chicken antibodies.

Humanized IgG monoclonal antibodies or human IgG monoclonal antibodies are preferable.

The anti-PD-1 antibody in the present disclosure is nivolumab, pembrolizumab, zimberelimab, cemiplimab, spartalizumab, budigalimab, camrelizumab, cetrelimab, dostarlimab, czabenlimab, lambrolizumab, pempulimab, pimivalimab, pucotenlimab, sasanlimab, sintilimab, tislelizumab, toripalimab or the like, preferably nivolumab, pembrolizumab or zimberclimab.

The anti-PD-L1 antibody in the present disclosure is atezolizumab, durvalumab, avelumab, garivulimab, lodapolimab, cosibelimab, socazolimab or the like, preferably atczolizumab, durvalumab or avelumab, more preferably atezolizumab.

The anti-CTLA-4 antibody in the present disclosure is ipilimumab, tremelimumab, quavonlimab, zalifrelimab or the like, preferably ipilimumab.

The CTLA-4-Ig in the present disclosure is abatacept or the like, preferably abatacept.

The anti-TIGIT antibody in the present disclosure is vibostolimab, ociperlimab, domvanalimab, tiragolumab or the like.

The anti-LAG-3 antibody in the present disclosure is relatlimab, favezelimab, fianlimab or the like.

These antibodies can be normally produced by a known method for preparation of an antibody. Moreover, a commercially available antibody can also be used.

In the present disclosure, when two or more immune checkpoint molecule regulators are used, for example, the anti-PD-1 antibody and the anti-CTLA-4 antibody or the anti-PD-1 antibody and the anti-LAG-3 antibody can be used in combination, or a bispecific antibody capable of being bound to both PD-1 and CTLA-4 or both PD-1 and LAG-3 can be used. Examples of the bispecific antibody include XmAb20717 (PD-1×CTLA-4) and Tebotelimab (PD-1×LAG-3).

In the present disclosure, the dose per dosing day of the Compound A or a salt thereof is preferably 50 to 200%, more preferably 75 to 150%, particularly preferably 100%, of the recommended dosage of the singly administered Compound A or salt thereof, from the viewpoint of the enhancing effect on the antitumor effect of the immune checkpoint molecule regulator by Compound A.

A typical daily dose of Compound A or salt thereof can be in the range from 100 picograms to 100 milligrams per kilogram of body weight, more typically 10 nanograms to 25 milligrams per kilogram of bodyweight. More typically, a daily dose of Compound A or salt thereof can be in the range from 100 nanograms to 20 milligrams per kilogram of bodyweight although higher or lower doses may be administered where required. For example, the daily dose may be 1 micrograms to 20 milligrams of bodyweight, more typically 10 micrograms to 20 milligrams per kilogram of bodyweight, and more typically 100 micrograms to 20 milligrams per kilogram of bodyweight.

Dosages may also be expressed as the amount of drug administered relative to the body surface area of the patient (mg/m²). A typical daily dose of Compound A or salt thereof can be in the range from 3700 pg/m² to 3700 mg/m², although higher or lower doses may be administered where required. For example, the daily dose may be 370 ng/m² to 925 mg/m², more typically 3700 ng/m² to 740 mg/m², although higher or lower doses may be administered where required. For example, 37 micrograms/m² to 740 mg/m², and more typically 370 micrograms/m² to 740 mg/m², or 3700 micrograms/m² to 740 mg/m².

Compound A or salt thereof of the disclosure may be administered orally in a range of single doses, for example 0.05 to 3000 mg. Typically, the range may be 10 to 1000 mg. Typical examples of doses include 10, 20, 30, 40, 50, 60, 70, 80, 90, 100, 150, 200, 250, 300, 350, 400, 450, 500, 600, 700, 800, 900 and 1000 mg. The doses may be increased or decreased in a stepwise manner from any dose in the above range (of 0.05 to 3000 mg) in increments/decrements of, for example, 1 mg, 5 mg, 10 mg, 20 mg, or 50 mg.

In the present disclosure, the dose per dosing day of the immune checkpoint molecule regulator is preferably 50 to 200%, more preferably 75 to 150%, particularly preferably 100% of the recommended dosage of the singly administered immune checkpoint molecule regulator, from the viewpoint of the enhancing action on the antitumor effect of the immune checkpoint molecule regulator by Compound A.

Typical example of the recommended dosage of singly administered nivolumab is 2 mg/kg (body weight) per administration or 3 mg/kg (body weight) per administration.

Typical example of the recommended dosage of singly administered pembrolizumab is 2 mg/kg (body weight) per administration or 200 mg per administration.

Typical example of the recommended dosage of singly administered atezolizumab is 1200 mg per administration.

Typical example of the recommended dosage of singly administered avelumab or durvalumab is 10 mg/kg (body weight) per administration.

Typical example of the recommended dosage of singly administered ipilimumab is 3 mg/kg (body weight) per administration.

In the present disclosure, the “recommended dosage” is a dose which is determined through a clinical test etc. and at which a maximum therapeutic effect is produced while safe use can be ensured without development of severe side-effects. Specifically, the “recommended dosage” is a dosage approved, recommended or suggested by public organizations or corporations such as Pharmaceuticals and Medical Devices Agency (PMDA), Food and Drug Administration (FDA) and European Medicines Agency (EMA), and described in attached documents, interview forms, treatment guidelines or the like, preferably a dose approved by a public organization selected from the group consisting of PMDA, FDA and EMA.

The administration schedule of the antitumor agent of the present disclosure can be appropriately selected according to the type of cancer, the stage of disease, etc.

The administration schedule of Compound A or a salt thereof is not particularly limited, as long as it includes a one or two week period of continuous administration, followed by a resting period of one week. When a two week (14 day) administration schedule, consisting of one week (7 days) of continuous administration followed by a one week (7 day) resting period is defined as one (1) cycle, the cycle can be performed once or repeated twice or more to treat the disease or disorder. That is, the administration can be carried out in one cycle, or more than one cycle, for example, 1, 2, 3, 4, 5, 6, 7, 8, 9, 10 cycles or more of a specific administration schedule. In some embodiments, the administration can be carried out in long-periods comprising several cycles. For example, the administration can be carried out for a period of 6-months with approximately 13 to 15 cycles of treatment or administration; a 1-year period with approximately 25 or 26 cycles of treatment; a 3-year period with approximately 75 to 100 cycles of treatment or more. When a three week (21 day) administration schedule, consisting of two weeks (14 days) of administration followed by a one week (7 day) resting period, is defined as one (1) cycle, the cycle can be performed once or repeated two or more times. That is, the administration can be carried out in one cycle, or more than one cycle, for example, 1, 2, 3, 4, 5, 6, 7, 8, 9, 10 cycles, or more of the administration schedule. In some cases, the administration can be carried out in more cycles. For example, the administration can be carried out for a period of 6 months with approximately 10 cycles; a 1-year period with approximately 20 cycles; a 3-year period with approximately 50 to 60 cycles or longer periods of administration with more cycles.

Moreover, in the administration schedule of Compound A, as long as the administration is continued on the schedule of a one or two week period of continuous administration, followed by a resting period of one week, the administration may be stopped thereafter, and the administration may be restarted after a certain period of drug holiday (no administration). Similarly, the administration schedule of the present disclosure may include a schedule having a plurality of periods of drug holiday. In one embodiment of an administration schedule having a drug holiday, it is sufficient that the conditions “one or two week continuous administration followed by a resting period of one week” are satisfied in the dosing period before the drug holiday and in the dosing period after the drug holiday. In another embodiment of an administration schedule having two periods of drug holiday, it is sufficient that the conditions “one or two week continuous administration followed by a resting period of one week” are satisfied in the dosing period before the first period of drug holiday, in the dosing period between the two periods of drug holiday, and in the dosing period after the second period of drug holiday. In another embodiment of an administration schedule having two or more periods of drug holiday, it is sufficient that the conditions “one or two week continuous administration followed by a resting period of one week” are satisfied in the dosing period before the first period of drug holiday, in the dosing period between the two adjacent periods of drug holiday, and in the dosing period after the last period of drug holiday. The period of drug holiday is not particularly limited, and can be suitably set according to the patient's state, and the like. For example, the period of drug holiday can be within the range of 1 to 35 days. Alternatively, the period of drug holiday can be within the range of 1 to 12 months.

The administration schedule of the immune checkpoint molecule regulator is also not particularly limited as long as administered at a certain interval. In some embodiments, the immune checkpoint molecule regulator is administered at intervals of 1 to 3 weeks.

The administration schedule of nivolumab is preferably an administration schedule in which administration is performed at intervals of 2 or 3 weeks.

The administration schedule of pembrolizumab, atezolizumab or ipilimumab is preferably an administration schedule in which administration is performed at intervals of 3 weeks.

The administration schedule of avelumab or durvalumab is preferably an administration schedule in which administration is performed at intervals of 2 weeks.

The number of doses per day of the antitumor agent of the present disclosure is appropriately selected according to the type of cancer, the stage of disease, etc.

The number of doses per day of Compound A or a salt thereof is preferably 1 or 2, more preferably 1. The number of doses per day of nivolumab, pembrolizumab, atczolizumab, avelumab, durvalumab or ipilimumab is preferably 1.

The order of administration of Compound A or a salt thereof and the immune checkpoint molecule regulator in the present disclosure can be appropriately selected according to the type of cancer, the stage of disease, etc., and these agents may be administered in any order, or in parallel when they are used in combination.

“in combination” can mean use of one agent in a treatment of a subject, wherein the treatment comprises administering another agent to the subject.

Here, when Compound A or a salt thereof and the immune checkpoint molecule regulator are not administered in parallel, the administration interval between the two agents can be appropriately selected as long as an enhancing effect on the antitumor effect is exhibited, and the administration interval is preferably 1 to 14 days, more preferably 1 to 7 days, still more preferably 1 to 5 days, particularly preferably 1 to 3 days.

The tumor to be targeted in the present disclosure is not particularly limited as long as an enhancing effect on the antitumor effect is exhibited, and the tumor is preferably a tumor against which Compound A or a salt thereof exhibits an antitumor effect, more preferably a malignant tumor in which LSD1 is involved.

The type of malignant tumor to be treated by the Compound A or a salt thereof is not particularly limited. Examples of such malignant tumors include glandular tumors, carcinoid tumors, undifferentiated carcinomas, angiosarcoma, adenocarcinoma, gastrointestinal cancers (e.g., colorectal cancers (“CRC”) including colon cancer and rectal cancer, biliary cancers including gall bladder cancer and bile duct cancer (cholangiocarcinoma), anal cancer, esophageal cancer, gastric (stomach) cancer, gastrointestinal carcinoid tumor(s), gastrointestinal stromal tumor(s) (“GIST”), liver cancer, duodenal cancer and small intestine cancer), lung cancers (e.g., non-small cell lung cancer (“NSCLC”), squamous-cell lung carcinoma, large-cell lung carcinoma, small cell lung carcinoma, invasive mucinous adenocarcinoma, mesothelioma and other lung cancers such as bronchial tumors and pleuropulmonary blastoma), urological cancers (e.g., kidney (renal) cancer, transitional cell cancer (“TCC”) of kidney, TCC of the renal pelvis and ureter (“PDQ”), bladder cancer, urethral cancer and prostate cancer), head and neck cancers (e.g., eye cancer, retinoblastoma, intraocular melanoma, hypopharyngeal cancer, pharyngeal cancer, laryngeal cancer, laryngeal papillomatosis, metastatic squamous neck cancer with occult primary, sinonasal squamous cell carcinoma (SNSCC), oral (mouth) cancer, lip cancer, throat cancer, oropharyngeal cancer, esthesioneuroblastoma, nasal cavity and paranasal sinus cancer, nasopharyngeal cancer, and salivary gland cancer), endocrine cancers (e.g., thyroid cancer, parathyroid cancer, multiple endocrine neoplasia syndromes, thymoma and thymic carcinoma, pancreatic cancers including pancreatic ductal adenocarcinoma (“PDAC”), pancreatic neuroendocrine tumors and islet cell tumors), breast cancers (extrahepatic ductal carcinoma in situ (“DCIS”), lobular carcinoma in situ (“LCIS”), triple negative breast cancer, and inflammatory breast cancer), male and female reproductive cancers (e.g., cervical cancer, ovarian cancer, endometrial cancer, uterine sarcoma, uterine cancer, vaginal cancer, vulvar cancer, gestational trophoblastic tumor (“GTD”), extragonadal germ cell tumor, extracranial germ cell tumor, germ cell tumor, testicular cancer and penile cancer), brain and nervous system cancers (e.g., astrocytomas, brain stem glioma, brain tumor, glioblastoma (GBM), craniopharyngioma, central nervous system (“CNS”) cancer, chordomas, ependymoma, embryonal tumors, neuroblastoma, paraganglioma, atypical teratoid, oligodendroma, oligodendroastrocytoma, oligodendroglioma, anaplastic oligodendroastrocytoma, ganglioglioma, central neurocytoma, medulloblastoma, germinoma, meningioma, neurilemmoma, GH secreting pituitary adenoma, PRL-secreting pituitary adenoma, ACTH-secreting pituitary adenoma, nonfunctional pituitary adenoma, hemangioblastoma, and epidermoid tumor), skin cancers (e.g., basal cell carcinoma (“BCC”), squamous cell skin carcinoma (“SCC”), Merkel cell carcinoma and melanoma), tissue and bone cancers (e.g., soft-tissue sarcoma, rhabdomyosarcoma, fibrous histiocytoma of bone, Ewing sarcoma, malignant fibrous histiocytoma of bone (“MFH”), osteosarcoma and chondrosarcoma), cardiovascular cancers (e.g., heart cancer and cardiac tumors), appendix cancers, childhood and adolescent cancers (e.g., adrenocortical carcinoma childhood, midline tract carcinoma, hepatocellular carcinoma (“HCC”), hepatoblastoma and Wilms' tumor) and viral-induced cancers (e.g., HHV-8 related cancers (Kaposi sarcoma) and HIV/AIDS related cancers).

Tumors also suitable for treatment may include, but are not limited to, hematological and plasma cell malignancies (e.g., cancers that affect blood, bone marrow and/or lymph nodes) such as multiple myeloma, leukemias and lymphomas, myelodysplastic syndromes and myeloproliferative disorders. Leukemias include, without limitation, acute lymphoblastic leukemia (“ALL”), acute myelogenous (myeloid) leukemia (“AML”), chronic lymphocytic leukemia (“CLL”), chronic myelogenous leukemia (“CML”), acute monocytic leukemia (“AMoL”), hairy cell leukemia, and/or other leukemias. Lymphomas include, without limitation, Hodgkin's lymphoma and non-Hodgkin's lymphoma (“NHL”). In some embodiments, NHL is B-cell lymphomas and/or T-cell lymphomas. In some embodiments, NHL includes, without limitation, diffuse large B-cell lymphoma (“DLBCL”), small lymphocytic lymphoma (“SLL”), chronic lymphocytic leukemia (“CLL”), mantle cell lymphoma (“MCL”), Burkitt's lymphoma, cutaneous T-cell lymphoma including mycosis fungoides and Sezary syndrome, AIDS-related lymphoma, follicular lymphoma, lymphoplasmacytic lymphoma (Waldenstrom's macroglobulinemia (“WM”)), primary central nervous system (CNS) lymphoma, central nervous system malignant lymphoma, and/or other lymphomas.

More preferably, examples include lung cancers (non-small-cell lung cancer, small-cell lung cancer, etc.) and leukemia. More preferably, examples include small-cell lung cancer (SCLC) and acute myeloid leukemia (AML).

As shown in Examples below, Compound A or a salt thereof has immunostimulatory action. As used herein, the “immunostimulatory action” means the action of activating immune cells such as cytotoxic T cells and macrophages and/or the action of inhibiting the suppression of immune response by regulatory T cells, myeloid-derived suppressor cells (MDSC), etc. In particular, Compound A or a salt thereof has the action of increasing cytotoxic T cells and/or the action of inhibiting the suppression of immune response by granulocytic myeloid-derived suppressor cells (gMDSC).

In the present disclosure, gMDSC can be identified by markers conventionally known to persons skilled in the art. Examples of the marker include CD33⁺CD11b⁺HLA-DR⁻CD15⁺CD14⁻, CD33⁺CD11b⁺HLA-DR^{low}CD15⁺CD14⁻, CD33⁺CD11b⁺Lin⁻ CD15⁺CD14⁻, CD33⁺CD11b⁺HLA-DR⁻Lin⁻CD15⁺CD14⁻ and CD33⁺CD11b⁺HLA-DR^{low}Lin⁻CD15⁺CD14⁻.

In the present disclosure, active ingredients Compound A or a salt thereof and the immune checkpoint molecule regulator may be separated and formulated in a plurality of dosage forms according to the formulations or administration schedules of the active ingredients, or integrated and formulated in one dosage form (i.e. formulated as a combination preparation). The preparations may be produced and sold in one package suitable for combination administration, or produced and sold in separate packages.

The formulations of the antitumor agent according to the present disclosure are not particularly limited, and can be appropriately selected according to the treatment purpose, and specific examples thereof include oral preparations (tablets, coated tablets, powders, granules, capsules, solutions, etc.), injections, suppositories, patches and ointments.

In the case of Compound A or a salt thereof, oral preparations are preferable.

In the case of the anti-PD-1 antibody, anti-PD-L1 antibody or anti-CTLA-4 antibody, the aforementioned formulations are used, with injections being preferable.

For both Compound A or a salt thereof and the immune checkpoint molecule regulator, the antitumor agent according to the present disclosure can be normally prepared by a known method using one or more pharmaceutical acceptable carriers depending on the formulations of the agents. Examples of the carriers include various carriers which are commonly used for normal pharmaceutical agents, e.g. excipients, binders, disintegrants, lubricants, diluents, solubilizers, suspending agents, tonicity agents, pH adjustors, buffers, stabilizers, coloring agents, flavor improving agents and odor improving agents.

The antitumor agent of the present disclosure makes it possible to perform cancer treatment with high antitumor effect while suppressing development of side-effects. In some embodiments, the high antitumor effect include a tumor growth delaying effect, a tumor reducing effect, or both. Thus, when the antitumor agent is administered to a cancer patient, the life of the patient is extended.

The present disclosure also relates to an antitumor effect enhancing agent for enhancing the antitumor effect of an immune checkpoint molecule regulator on a cancer patient, the antitumor effect enhancing agent containing Compound A or a salt thereof as an active ingredient. The antitumor effect enhancing agent has a preparation form identical to that of the aforementioned antitumor agent.

The present disclosure also relates to an antitumor effect enhancing agent for enhancing the antitumor effect of Compound A or a salt thereof on a cancer patient, the antitumor effect enhancing agent containing an immune checkpoint molecule regulator as an active ingredient. The antitumor effect enhancing agent has a preparation form identical to that of the aforementioned antitumor agent.

The present disclosure also relates to an antitumor agent for treating a cancer patient given an immune checkpoint molecule regulator, the antitumor agent containing Compound A or a salt thereof. The antitumor agent has the aforementioned preparation form.

The present disclosure also relates to an antitumor agent for treating a cancer patient given Compound A or a salt thereof, the antitumor agent containing an immune checkpoint molecule regulator. The antitumor agent has the aforementioned preparation form.

The “treatment” includes postoperative adjuvant chemotherapy to be carried out for prevention of recurrence after surgical removal of tumor, and preoperative adjuvant chemotherapy to be carried out before surgical removal of tumor.

The present disclosure also relates to an antitumor agent containing Compound A or a salt thereof, used in combination with an immune checkpoint molecule regulator for a cancer patient. The antitumor agent has the aforementioned preparation form.

The present disclosure also relates to an antitumor agent containing an immune checkpoint molecule regulator, used in combination with an antitumor agent containing Compound A or a salt thereof for a cancer patient. The antitumor agent has the aforementioned preparation form.

The present disclosure also relates to a kit preparation including an antitumor agent containing Compound A or a salt thereof; and a written instruction which indicates that Compound A or a salt thereof and an immune checkpoint molecule regulator are administered in combination to a cancer patient.

Here, the “written instruction” is not limited as long as the aforementioned doses are specified. The written instruction may be legally binding or non-binding, and is preferably one in which the aforementioned doses are recommended. Specific examples of the written instruction include attached documents and brochures. The kit preparation including a written instruction may be one in which a written instruction is printed or attached on a kit preparation package, or one in which a written instruction is enclosed together with an antitumor agent in a kit preparation package.

The present disclosure may be further combined with one or more anticancer agents other than the antitumor agents described above. Examples of anticancer agents include chemotherapeutic agents (e.g., cytotoxic agents), immunotherapeutic agents, hormonal and anti-hormonal agents, targeted therapy agents, and anti-angiogenesis agents. Many anti-cancer agents can be classified within one or more of these groups. While certain anticancer agents have been categorized within a specific group(s) or subgroup(s) herein, many of these agents can also be listed within one or more other group(s) or subgroup(s), as would be presently understood in the art. The anticancer agent is not particularly limited, and examples thereof include, but are not limited to, a chemotherapeutic agent, a mitotic inhibitor, a plant alkaloid, an alkylating agent, an anti-metabolite, a platinum analog, an enzyme, a topoisomerase inhibitor, a retinoid, an aziridine, an antibiotic, a hormonal agent, an anti-hormonal agent, an anti-estrogen, an anti-androgen, an anti-adrenal, an androgen, a targeted therapy agent, an immunotherapeutic agent, a biological response modifier, a cytokine inhibitor, a tumor vaccine, a monoclonal antibody, a colony-stimulating factor, an anti-LAG1 agent, an anti-OX40 agent.

Non-limiting examples of chemotherapeutic agents include mitotic inhibitors, plant alkaloids, alkylating agents, anti-metabolites, platinum analogs, enzymes, topoisomerase inhibitors, retinoids, aziridines, and antibiotics.

Non-limiting examples of mitotic inhibitors and plant alkaloids include taxanes such as cabazitaxel, docetaxel, larotaxel, ortataxel, paclitaxel, and tesetaxel; demecolcine; epothilone; eribulin; etoposide (VP-16); etoposide phosphate; navelbine; noscapine; teniposide; thaliblastine; vinblastine; vincristine; vindesine; vinflunine; and vinorelbine.

Non-limiting examples of alkylating agents include nitrogen mustards such as chlorambucil, chlornaphazine, cholophosphamide, cytophosphane, estramustine, ifosfamide, mannomustine, mechlorethamine, mechlorethamine oxide hydrochloride, melphalan, novembichin, phenesterine, prednimustine, tris(2-chloroethyl)amine, trofosfamide, and uracil mustard; alkyl sulfonates such as busulfan, improsulfan, and piposulfan; nitrosoureas such as carmustine, chlorozotocin, fotemustine, lomustine, nimustine, ranimustine, streptozotocin, and TA-07; ethylenimines and methylamelamines such as altretamine, thiotepa, triethylenemelamine, triethylenethiophosphaoramide, trictylenephosphoramide, and trimethylolomelamine; ambamustine; bendamustine; dacarbazine; cyclophosphamide; etoglucid; irofulven; mafosfamide; mitobronitol; mitolactol; pipobroman; procarbazine; temozolomide; treosulfan; and triaziquone.

Non-limiting examples of anti-metabolites include folic acid analogues such as aminopterin, denopterin, edatrexate, methotrexate, pteropterin, raltitrexed, and trimetrexate; purine analogs such as 6-mercaptopurine, 6-thioguanine, fludarabine, forodesine, thiamiprine, and thioguanine; pyrimidine analogs such as 5-fluorouracil (5-FU), tegafur/gimeracil/oteracil potassium, tegafur/uracil, trifluridine, trifluridine/tipiracil hydrochloride, 6-azauridine, ancitabine, azacytidine, capecitabine, carmofur, cytarabine, decitabine, dideoxyuridine, doxifiuridine, doxifluridine, enocitabine, floxuridine, galocitabine, gemcitabine, and sapacitabine;

3-aminopyridine-2-carboxaldehyde thiosemicarbazone; broxuridine; cladribine; cyclophosphamide; cytarabine; emitefur; hydroxyurea; mercaptopurine; nelarabine; pemetrexed; pentostatin; tegafur; and troxacitabine.

Non-limiting examples of platinum analogs include carboplatin, cisplatin, dicycloplatin, heptaplatin, lobaplatin, nedaplatin, oxaliplatin, satraplatin, and triplatin tetranitrate.

Non-limiting examples of enzymes include asparaginase and pegaspargase.

Non-limiting examples of topoisomerase inhibitors include acridine carboxamide, amonafide, amsacrine, belotecan, elliptinium acetate, exatecan, indolocarbazole, irinotecan, lurtotecan, mitoxantrone, razoxane, rubitecan, SN-38, sobuzoxane, and topotecan.

Non-limiting examples of retinoids include alitretinoin, bexarotene, fenretinide, isotretinoin, liarozole, RII retinamide, and tretinoin.

Non-limiting examples of aziridines include benzodopa, carboquone, meturedopa, and uredopa.

Non-limiting examples of antibiotics include intercalating antibiotics; anthracenediones; anthracycline antibiotics such as aclarubicin, amrubicin, daunomycin, daunorubicin, doxorubicin, epirubicin, idarubicin, menogaril, nogalamycin, pirarubicin, and valrubicin; 6-diazo-5-oxo-L-norleucine; aclacinomysins; actinomycin; authramycin; azaserine; bleomycins; cactinomycin; calicheamicin; carabicin; carminomycin; carzinophilin; chromomycins; dactinomycin; detorubicin; esorubicin; esperamicins; geldanamycin; marcellomycin; mitomycins; mitomycin C; mycophenolic acid; olivomycins; novantrone; peplomycin; porfiromycin; potfiromycin; puromycin; quelamycin; rebeccamycin; rodorubicin; streptonigrin; streptozocin; tanespimycin; tubercidin; ubenimex; zinostatin; zinostatin stimalamer; and zorubicin.

Non-limiting examples of hormonal and anti-hormonal agents include anti-androgens such as abiraterone, apalutamide, bicalutamide, darolutamide, enzalutamide, flutamide, goserelin, leuprolide, and nilutamide; anti-estrogens such as 4-hydroxy tamoxifen, aromatase inhibiting 4 (5)-imidazoles, EM-800, fosfestrol, fulvestrant, keoxifene, LY 117018, onapristone, raloxifene, tamoxifen, toremifene, and trioxifene; anti-adrenals such as aminoglutethimide, dexaminoglutethimide, mitotane, and trilostane; androgens such as calusterone, dromostanolone propionate, epitiostanol, mepitiostane, and testolactone; abarelix; anastrozole; cetrorelix; deslorelin; exemestane; fadrozole; finasteride; formestane; histrelin (RL 0903); human chorionic gonadotropin; lanreotide; LDI 200 (Milkhaus); letrozole; leuprorelin; mifepristone; nafarelin; nafoxidine; osaterone; prednisone; thyrotropin alfa; and triptorelin.

Non-limiting examples of immunotherapeutic agents (i.e., immunotherapy) include biological response modifiers, cytokine inhibitors, tumor vaccines, monoclonal antibodies, colony-stimulating factors, and immunomodulators.

Non-limiting examples of biological response modifiers, including cytokine inhibitors (cytokines) such as interferons and interleukins, include interferon alfa/interferon alpha such as interferon alfa-2, interferon alfa-2a, interferon alfa-2b, interferon alfa-n1, interferon alfa-n3, interferon alfacon-1, peginterferon alfa-2a, peginterferon alfa-2b, and leukocyte alpha interferon; interferon beta such as interferon beta-1a, and interferon beta-1b; interferon gamma such as natural interferon gamma-1a, and interferon gamma-1b; aldesleukin; interleukin-1 beta; interleukin-2; oprelvekin; sonermin; tasonermin; and virulizin.

Non-limiting examples of tumor vaccines include APC 8015, AVICINE, bladder cancer vaccine, cancer vaccine (Biomira), gastrin 17 immunogen, Maruyama vaccine, melanoma lysate vaccine, melanoma oncolysate vaccine (New York Medical College), melanoma vaccine (New York University), melanoma vaccine (Sloan Kettering Institute), TICE® BCG (Bacillus Calmette-Guerin), and viral melanoma cell lysates vaccine (Royal Newcastle Hospital).

Non-limiting examples of monoclonal antibodies include abagovomab, adecatumumab, aflibercept, alemtuzumab, blinatumomab, brentuximab vedotin, CA 125 MAb (Biomira), cancer MAb (Japan Pharmaceutical Development), daclizumab, daratumumab, denosumab, edrecolomab, gemtuzumab zogamicin, HER-2 and Fc MAb (Medarex), ibritumomab tiuxetan, idiotypic 105AD7 MAb (CRC Technology), idiotypic CEA MAb (Trilex), lintuzumab, LYM-1-iodine 131 MAb (Techni clone), mitumomab, moxetumomab, ofatumumab, polymorphic epithelial mucin-yttrium 90 MAb (Antisoma), ranibizumab, rituximab, veltuzumab, and trastuzumab.

Non-limiting examples of colony-stimulating factors include darbepoetin alfa, epoetin alfa, epoetin beta, filgrastim, granulocyte macrophage colony stimulating factor, lenograstim, leridistim, mirimostim, molgramostim, nartograstim, pegfilgrastim, and sargramostim.

Non-limiting examples of additional immunotherapeutic agents include BiTEs, CAR-T cells, GITR agonists, imiquimod, immunomodulatory imides (IMiDs), mismatched double stranded RNA (Ampligen), resiquimod, SRL 172, and thymalfasin.

Targeted therapy agents include, for example, monoclonal antibodies and small molecule drugs. Non-limiting examples of targeted therapy agents include signal transduction inhibitors, growth factor inhibitors, tyrosine kinase inhibitors, EGFR inhibitors, HER2 inhibitors, histone deacetylase (HDAC) inhibitors, proteasome inhibitors, cell-cycle inhibitors, angiogenesis inhibitors, matrix-metalloproteinase (MMP) inhibitors, hepatocyte growth factor inhibitors, TOR inhibitors, KDR inhibitors, VEGF inhibitors, fibroblast growth factors (FGF) inhibitors, RAF inhibitor, MEK inhibitors, ERK inhibitors, PI3K inhibitors, AKT inhibitors, MCL-1 inhibitors, BCL-2 inhibitors, SHP2 inhibitors, BRAF-inhibitors, RAS inhibitor, gene expression modulators, autophagy inhibitors, apoptosis inducers, antiproliferative agents, and glycolysis inhibitors.

Non-limiting examples of signal transduction inhibitors include tyrosine kinase inhibitors, multiple-kinase inhibitors, anlotinib, avapritinib, axitinib, dasatinib, dovitinib, imatinib, lenvatinib, lonidamine, nilotinib, nintedanib, pazopanib, pegvisomant, ponatinib, vandetanib, and EGFR and/or HER2 inhibitory agents.

Non-limiting examples of EGFR inhibitors include small molecule antagonists of EGFR such as afatinib, brigatinib, erlotinib, gefitinib, lapatinib, neratinib, dacomitinib, vandetanib, and osimertinib; and antibody-based EGFR inhibitors, including any anti-EGFR antibody or antibody fragment that can partially or completely block EGFR activation by its natural ligand. Antibody-based EGFR inhibitory agents may include, for example, those described in Modjtahedi, H., et al., 1993, Br. J. Cancer 67:247-253; Teramoto, T., et al., 1996, Cancer 77:639-645; Goldstein et al, 1995, Clin. Cancer Res. 1:1311-1318; Huang, S. M., et al., 1999, Cancer Res. 15:59 (8): 1935-40; and Yang, X., et al., 1999, Cancer Res. 59:1236-1243; monoclonal antibody Mab E7.6.3 (Yang, 1999 supra); Mab C225 (ATCC Accession No. HB-8508), or an antibody or antibody fragment having the binding specificity thereof; specific antisense nucleotide or siRNA; afatinib, cetuximab; matuzumab; necitumumab; nimotuzumab; panitumumab; and zalutumumab.

Non-limiting examples of HER2 inhibitors include HER2 tyrosine kinase inhibitors such as afatinib, lapatinib, neratinib, and tucatinib; and anti-HER2 antibodies or drug conjugates thereof such as trastuzumab, trastuzumab emtansine (T-DM1), pertuzumab, margetuximab, trastuzumab deruxtecan (DS-8201a), and trastuzumab duocarmazine.

Non-limiting examples of histone deacetylase (HDAC) inhibitors include belinostat, panobinostat, romidepsin, and vorinostat.

Non-limiting examples of proteasome inhibitors include bortezomib, carfilzomib, ixazomib, marizomib (salinosporamide a), and oprozomib.

Non-limiting examples of cell-cycle inhibitors, including CDK inhibitors, include abemaciclib, alvocidib, palbociclib, and ribociclib.

Non-limiting examples of anti-angiogenic agents (or angiogenesis inhibitors) include, but not limited to, matrix-metalloproteinase (MMP) inhibitors; VEGF inhibitors; EGFR inhibitors; TOR inhibitors such as everolimus and temsirolimus; PDGFR kinase inhibitory agents such as crenolanib; HIF-1α inhibitors such as PX 478; HIF-2α inhibitors such as belzutifan and the HIF-2α inhibitors described in WO 2015/035223; fibroblast growth factor (FGF) or FGFR inhibitory agents such as BFGF and RG 13577; hepatocyte growth factor inhibitors; KDR inhibitors; anti-Ang1 and anti-Ang2 agents; anti-Tie2 kinase inhibitory agents; Tek antagonists (US 2003/0162712; U.S. Pat. No. 6,413,932); anti-TWEAK agents (U.S. Pat. No. 6,727,225); ADAM distintegrin domain to antagonize the binding of integrin to its ligands (US 2002/0042368); anti-eph receptor and/or anti-ephrin antibodies or antigen binding regions (U.S. Pat. Nos. 5,981,245; 5,728,813; 5,969,110; 6,596,852; 6,232,447; and 6,057,124); and anti-PDGF-BB antagonists as well as antibodies or antigen binding regions specifically binding to PDGF-BB ligands.

Non-limiting examples of matrix-metalloproteinase (MMP) inhibitors include MMP-2 (matrix-metalloproteinase 2) inhibitors, MMP-9 (matrix-metalloproteinase 9) inhibitors, prinomastat, RO 32-3555, and RS 13-0830. Examples of useful matrix metalloproteinase inhibitors are described, for example, in WO 96/33172, WO 96/27583, EP 1004578, WO 98/07697, WO 98/03516, WO 98/34918, WO 98/34915, WO 98/33768, WO 98/30566, EP 0606046, EP 0931788, WO 90/05719, WO 99/52910, WO 99/52889, WO 99/29667, WO 1999/007675, EP 1786785, EP 1181017, US 2009/0012085, U.S. Pat. Nos. 5,863,949, 5,861,510, and EP 0780386. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13).

Non-limiting examples of VEGF and VEGFR inhibitory agents include bevacizumab, cediranib, CEP 7055, CP 547632, KRN 633, orantinib, pazopanib, pegaptanib, pegaptanib octasodium, semaxanib, sorafenib, sunitinib, VEGF antagonist (Borcan, Denmark), and VEGF-TRAP™.

Other anti-angiogenic agents may include, but are not limited to, 2-methoxyestradiol, AE 941, alemtuzumab, alpha-D148 Mab (Amgen, US), alphastatin, anecortave acetate, angiocidin, angiogenesis inhibitors, (SUGEN, US), angiostatin, anti-Vn Mab (Crucell, Netherlands), atiprimod, axitinib, AZD 9935, BAY RES 2690 (Bayer, Germany, BC 1 (Genoa Institute of Cancer Research, Italy), beloranib, benefin (Lane Labs, US), cabozantinib, CDP 791 (Celltech Group, UK), chondroitinase AC, cilengitide, combretastatin A4 prodrug, CP 564959 (OSI, US), CV247, CYC 381 (Harvard University, US), E 7820, EHT 0101, endostatin, enzastaurin hydrochloride, ER-68203-00 (IVAX, US), fibrinogen-E fragment, Flk-1 (ImClone Systems, US), forms of FLT 1 (VEGFR 1), FR-111142, GCS-100, GW 2286 (GlaxoSmithKline, UK), IL-8, ilomastat, IM-862, irsogladine, KM-2550 (Kyowa Hakko, Japan), lenalidomide, lenvatinib, MAb alpha5beta3 integrin, second generation (Applied Molecular Evolution, USA and MedImmune, US), MAb VEGF (Xenova, UK), marimastat, maspin (Sosci, Japan), metastatin, motuporamine C, M-PGA, ombrabulin, OXI4503, PI 88, platelet factor 4, PPI 2458, ramucirumab, rBPI 21 and BPI-derived antiangiogenic (XOMA, US), regorafenib, SC-236, SD-7784 (Pfizer, US), SDX 103 (University of California at San Diego, US), SG 292 (Telios, US), SU-0879 (Pfizer, US), TAN-1120, TBC-1635, tesevatinib, tetrathiomolybdate, thalidomide, thrombospondin 1 inhibitor, Tie-2 ligands (Regeneron, US), tissue factor pathway inhibitors (EntreMed, US), tumor necrosis factor-alpha inhibitors, tumstatin, TZ 93, urokinase plasminogen activator inhibitors, vadimezan, vandetanib, vasostatin, vatalanib, VE-cadherin-2 antagonists, xanthorrhizol, XL 784 (Exclixis, US), ziv-aflibercept, and ZD 6126.

The anticancer agent(s) that may be combined with Compound A may also be an active agent that disrupts or inhibits RAS-RAF-ERK or PI3K-AKT-TOR signaling pathways. Examples of which include, but are not limited to, a RAF inhibitor, an EGFR inhibitor, a MEK inhibitor, an ERK inhibitor, a PI3K inhibitor, a AKT inhibitor, a TOR inhibitor, an MCL-1 inhibitor, a BCL-2 inhibitor, a SHP2 inhibitor, a proteasome inhibitor, or an immune therapy, including monoclonal antibodies, immunomodulatory imides (IMiDs), anti-LAG1, and anti-OX40 agents, GITR agonists, CAR-T cells, and BiTEs.

Non-limiting examples of RAF inhibitors include dabrafenib, encorafenib, regorafenib, sorafenib, and vemurafenib.

Non-limiting examples of MEK inhibitors include binimetinib, CI-1040, cobimetinib, PD318088, PD325901, PD334581, PD98059, refametinib, selumetinib, and trametinib.

Non-limiting examples of ERK inhibitors include LY3214996, LTT462, MK-8353, SCH772984, ravoxertinib, ulixertinib, and ASTX029.

Non-limiting examples of PI3K inhibitors include 17-hydroxywortmannin analogs (e.g., WO 06/044453); AEZS-136; alpelisib; AS-252424; buparlisib; CAL263; copanlisib; CUDC-907; dactolisib (WO 06/122806); demethoxyviridin; duvelisib; GNE477; GSK1059615; IC87114; idelalisib; INK1117; LY294002; Palomid 529; paxalisib; perifosine; PI-103; PI-103 hydrochloride; pictilisib (e.g., WO 09/036,082; WO 09/055,730); PIK 90; PWT33597; SF1126; sonolisib; TGI 00-115; TGX-221; XL147; XL-765; wortmannin; tasclisib (GDC-0032); and ZSTK474.

Non-limiting examples of AKT inhibitors include Akt-1-1 (inhibits Akt1) (Barnett et al. (2005) Biochem. J., 385 (Pt. 2), 399-408); Akt-1-1,2 (Barnett et al. (2005) Biochem. J. 385 (Pt. 2), 399-408); API-59CJ-Ome (e.g., Jin et al. (2004) Br. J. Cancer 91, 1808-12); 1-H-imidazo[4,5-c]pyridinyl compounds (e.g., WO05011700); indole-3-carbinol and derivatives thereof (e.g., U.S. Pat. No. 6,656,963; Sarkar and Li (2004) J Nutr. 134 (12 Suppl), 3493S-3498S); perifosine, Dasmahapatra et al. (2004) Clin. Cancer Res. 10 (15), 5242-52, 2004); phosphatidylinositol ether lipid analogues (e.g., Gills and Dennis (2004) Expert. Opin. Investig. Drugs 13, 787-97); triciribine (Yang et al. (2004) Cancer Res. 64, 4394-9); imidazooxazone compounds including trans-3-amino-1-methyl-3-[4-(3-phenyl-5H-imidazo[1,2-c]pyrido[3,4-c][1,3]oxazin-2-yl)phenyl]-cyclobutanol hydrochloride (WO 2012/137870); afuresertib; capivasertib; 8-[4-(1-aminocyclobutyl)phenyl]-9-phenyl-1,2,4-triazolo[3,4-f][1,6] naphthyridin-3(2H)-one (MK2206) and pharmaceutically acceptable salts thereof; AZD5363; trans-3-amino-1-methyl-3-(4-(3-phenyl-5H-imidazo[1,2-c]pyrido[3,4-c][1,3]oxazin-2-yl)phenyl)cyclobutanol (TAS117) and pharmaceutically acceptable salts thereof; and patasertib.

Non-limiting examples of TOR inhibitors include deforolimus; ATP-competitive TORC1/TORC2 inhibitors, including PI-103, PP242, PP30, and Torin 1; TOR inhibitors in FKBP12 enhancer, rapamycins and derivatives thereof, including temsirolimus, everolimus, WO 9409010; rapalogs, e.g. as disclosed in WO 98/02441 and WO 01/14387, e.g. AP23573, AP23464, or AP23841; 40-(2-hydroxyethyl)rapamycin, 40-[3-hydroxy (hydroxymethyl)methylpropanoate]-rapamycin; 40-epi-(tetrazolyl)-rapamycin (also called ABT578); AZD8055; 32-deoxorapamycin; 16-pentynyloxy-32(S)-dihydrorapanycin, and other derivatives disclosed in WO 05/005434; derivatives disclosed in U.S. Pat. No. 5,258,389, WO 94/090101, WO 92/05179, U.S. Pat. Nos. 5,118,677, 5,118,678, 5,100,883, 5,151,413, 5,120,842, WO 93/111130, WO 94/02136, WO 94/02485, WO 95/14023, WO 94/02136, WO 95/16691, WO 96/41807, WO 96/41807 and U.S. Pat. No. 5,256,790; and phosphorus-containing rapamycin derivatives (e.g., WO 05/016252).

Non-limiting examples of MCL-1 inhibitors include AMG-176, MIK665, and S63845.

Non-limiting examples of SHP2 inhibitors include JAB-3068, RMC-4630, TNO155, SHP-099, RMC-4550, and SHP2 inhibitors described in WO 2019/167000, WO 2020/022323 and WO2021/033153.

Non-limiting examples of RAS inhibitors include AMG510 (sotorasib), MRTX849, LY3499446, JNJ-74699157 (ARS-3248), ARS-1620, ARS-853, RM-007, and RM-008.

Additional non-limiting examples of anticancer agents that may be suitable for use include, but are not limited to, 2-ethylhydrazide, 2,2′,2″-trichlorotricthylamine, ABVD, aceglatone, acemannan, aldophosphamide glycoside, alpharadin, amifostine, aminolevulinic acid, anagrelide, ANCER, ancestim, anti-CD22 immunotoxins, antitumorigenic herbs, apaziquone, arglabin, arsenic trioxide, azathioprine, BAM 002 (Novelos), bcl-2 (Genta), bestrabucil, biricodar, bisantrene, bromocriptine, brostallicin, bryostatin, buthionine sulfoximine, calyculin, cell-cycle nonspecific antineoplastic agents, celmoleukin, clodronate, clotrimazole, cytarabine ocfosfate, DA 3030 (Dong-A), defofamine, denileukin diftitox, dexrazoxane, diaziquone, dichloroacetic acid, dilazep, discodermolide, docosanol, doxercalciferol, edelfosine, eflornithine, EL532 (Elan), elfomithine, elsamitrucin, eniluracil, etanidazole, exisulind, ferruginol, folic acid replenisher such as frolinic acid, gacytosine, gallium nitrate, gimeracil/oteracil/tegafur combination (S-1), glycopine, histamine dihydrochloride, HIT diclofenac, HLA-B7 gene therapy (Vical), human fetal alpha fetoprotein, ibandronate, ibandronic acid, ICE chemotherapy regimen, imexon, iobenguane, IT-101 (CRLX101), laniquidar, LC 9018 (Yakult), leflunomide, lentinan, levamisole plus fluorouracil, lovastatin, lucanthone, masoprocol, melarsoprol, metoclopramide, miltefosine, miproxifene, mitoguazone, mitozolomide, mopidamol, motexafin gadolinium, MX6 (Galderma), naloxone plus pentazocine, nitracrine, nolatrexed, NSC 631570 octreotide (Ukrain), olaparib, P-30 protein, PAC-1, palifermin, pamidronate, pamidronic acid, pontosan polysulfate sodium, phenamet, picibanil, pixantrone, platinum, podophyllinic acid, porfimer sodium, PSK (Polysaccharide-K), rabbit antithymocyte polyclonal antibody, rasburiembodiment, retinoic acid, rhenium Re 186 etidronate, romurtide, samarium (153 Sm) lexidronam, sizofiran, sodium phenylacetate, sparfosic acid, spirogermanium, strontium-89 chloride, suramin, swainsonine, talaporfin, tariquidar, tazarotene, tegafur-uracil, temoporfin, tenuazonic acid, tetrachlorodecaoxide, thrombopoietin, tin ethyl etiopurpurin, tirapazamine, TLC ELL-12, tositumomab-iodine 131, trifluridine and tipiracil combination, troponin I (Harvard University, US), urethan, valspodar, verteporfin, zoledronic acid, and zosuquidar.

### EXAMPLES

The present disclosure will now be described in further detail by way of examples, which should not be construed as limiting the present disclosure, and those skilled in the art can make many modifications within the technical concept of the present disclosure.

### Example 1: Immunostimulatory Action of Biphenyl Compound in Colon Cancer Cell Transplanted Model

The mouse colon cancer cell strain MC38 was obtained from Dr. Yoshihiro Hayakawa (University of Toyama, Toyama, Japan). The MC38 cell strain was subjected to culture in an RPMI 1640 medium containing 10% fetal bovine serum and then to subculture in an incubator at 37° C. in the presence of 5% CO2 at a frequency of once or twice a week.

The MC38 cell suspension was transplanted subcutaneously at 1.5×10⁶ cells/0.1 ml to the right chest of 6-week-old C57BL/6JJcl mice (CLEA Japan, Inc.).

The tumor was grown until reaching a tumor volume (TV) of 80 to 230 mm³ after the transplantation. Degimatic Caliper was used for tumor diameter measurement. The major diameter and the minor diameter of the tumor were measured, and TV was calculated from the following equation.

TV (mm³)=major diameter (mm)×minor diameter (mm)×minor diameter (mm)/2

By a stratified random allocation method using TV as an index, 5 animals were allocated to each group. The day when grouping (n=5) was performed was defined as Day 0.

An appropriate amount of water for injection described in the Japanese Pharmacopeia was added for adjusting hypromellose concentration to 0.5 w/v %, and the mixture was then stirred with a stirrer to completely dissolve hypromellose, to prepare a 0.5% aqueous hypromellose solution. Benzoic acid salt of Compound A (in the section of Examples, a benzoic acid salt of the Compound A was used. Thus, “Compound A” hereinafter refers to a benzoic acid salt of the Compound A) was ground with an agate mortar, and then suspended to a predetermined concentration with the 0.5% aqueous hypromellose solution, and ultrasonic treatment was performed to obtain a homogeneous suspension. The suspension containing Compound A was orally administered once a day for 6 consecutive days at a dose of 25 mg/kg/day. The dosage is indicated based on the free form of the Compound A.

The anti-mouse PD-1 antibody (anti-mPD-1 Ab) was prepared by diluting anti-PD-1, CD279 (PD-1) monoclonal antibody (Clone: RMP1-14, Thermo Fisher Scientific) to a predetermined concentration with Dulbecco's Phosphate Buffered Saline (nacalai tesque, INC.) immediately before administration. On the first day of administration (day 1), the diluted product was intraperitoneally administered with the dose of 0.05 mg/body of the anti-mouse PD-1 antibody.

For detecting expression of immunity-related factors, flow cytometric analysis was performed for immunological monitoring. On Day 7, the tumor was sampled, and tumor infiltrating lymphocytes were then prepared using Tumor Dissociation Kit, mouse (Miltenyi Biotec). Thereafter, the isolated lymphocytes were stained with antibodies below, and then, analyzed using Flow Cytometer FACSVerse (BD Bioscience). For the staining, CD45 monoclonal antibody (30-F11) eFluor 450, eBioscience™ (Thermo Fisher Scientific), Brilliant Violet 510™ anti-mouse CD90.2 antibody (30-H2) (BioLegend), CD11b monoclonal antibody (M1/70) PE, eBioscience™ (Thermo Fisher Scientific), Ly-6G antibody anti-mouse (REA526), REAfinity™ (Miltenyi Biotec) and CD8a monoclonal antibody (53-6.7) FITC, cBioscience™ (Thermo Fisher Scientific) were used.

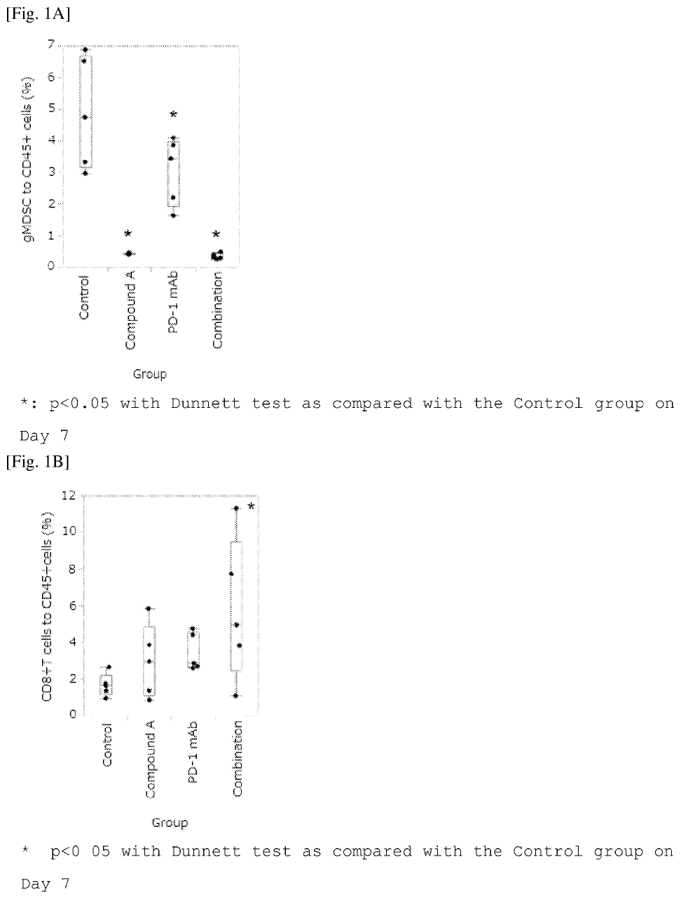

The results are shown in FIGS. 1A and 1B.

The ratio of gMDSC (CD45 positive, CD90.2 negative, CD11b positive, Ly6G positive cells) in leucocytes (CD45 positive cells) of each group on Day 7 was analyzed by the Dunnett test, and the result showed that the Compound A administered groups had a significantly lower ratio of gMDSC in leucocytes as compared to the control group on Day 7. The ratio of CD8 positive cells (CD45 positive, CD90.2 positive and CD8 positive cells) in leucocytes (CD45 positive cells) of each group on Day 7 was analyzed by the Dunnett test, and the result showed that the group treated with combination of Compound A and anti-mouse PD-1 antibody had a significantly higher ratio of CD8 positive cells in leucocytes as compared to the control group on Day 7.

The above results showed that Compound A shows immunostimulatory activity.

### Example 2: Effect of Biphenyl Compound in Colon Cancer Cell Transplanted Model

The MC38 cell suspension was transplanted subcutaneously at 2.0×10⁶ cells/0.1 ml to the right chest of 6-week-old C57BL/6JJcl mice (CLEA Japan, Inc.) or CB17/Icr-Prkdc[scid]/CrlCrlj (Charles River Laboratories Japan Inc.). CB17/Icr-Prkdc[scid]/CrlCrlj mice are mice with immune deficiency due to lack of Tcells and B-cells in the peripheral blood.

The tumor was grown until reaching a TV of 50 to 300 mm³ after the transplantation. Degimatic Caliper was used for tumor diameter measurement. The major diameter and the minor diameter of the tumor were measured, and TV was calculated from the following equation.

TV (mm³)=major diameter (mm)×minor diameter (mm)×minor diameter (mm)/2

By a stratified random allocation method using TV as an index, ten C57BL/6JJcl mice were allocated to each group, and five CB17/Icr-Prkdc [scid]/CrlCrlj mice were allocated to each group. The day when grouping was performed was defined as Day 0. An electronic balance for animals was used for body weight measurement. The body weight change ratio on the nth day (BWCn) was calculated from the body weight on the nth day (BWn) in accordance with the following equation.

Body weight change ratio BWCn (%)=(BWn−BW0)/BW0×100

An appropriate amount of water for injection described in the Japanese Pharmacopeia was added for adjusting hypromellose concentration to 0.5 w/v %, and the mixture was then stirred with a stirrer to completely dissolve hypromellose, to prepare a 0.5% aqueous hypromellose solution. Benzoic acid salt of Compound A was ground with an agate mortar, and then suspended to a predetermined concentration with the 0.5% aqueous hypromellose solution, and ultrasonic treatment was performed to obtain a homogeneous suspension. The suspension containing Compound A was orally administered once a day for 21 consecutive days at a dose of 25 mg/kg/day. The dosage is indicated based on the free form of the Compound A.

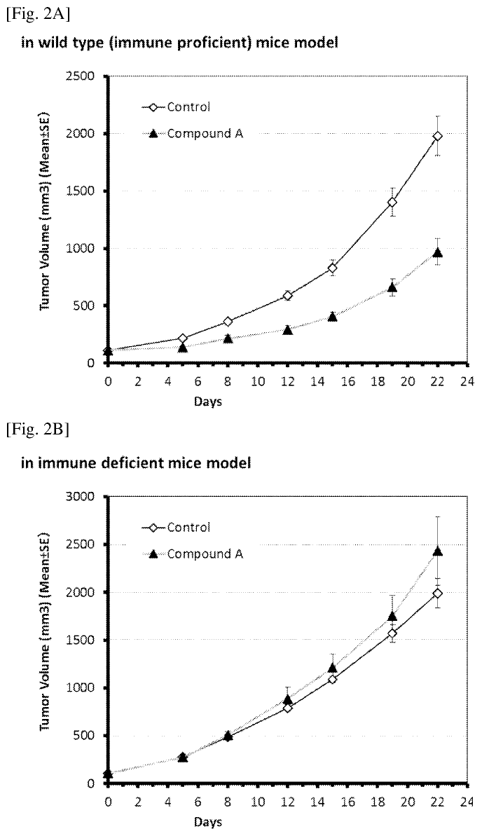

The results are shown in FIGS. 2A and 2B, Tables 1 and 2.

TABLE 1
Effect of Compound A in immune proficient, wild type (C57BL/6JJcl) mice

| | | | | Number | Tumor Volume (mm³) | | Body weight change (%) |
|---|---|---|---|---|---|---|---|
| | Dose | Dosing | | of | Day 22 | | Day 22 |
| Group | (mg/kg/day) | Schedule | Route | Animals | Mean ± SE | | Mean ± SE |
| Control | 0 | qd, Day 1-21 | po | 10 | 1980.4 ± 170.3 | | 5.8 ± 1.6 |
| Compound A | 25.0 | qd, Day 1-21 | po | 10 | 969.3 ± 114.7 | * | 2.6 ± 1.2 |

Abbreviations:
qd, quaque die (once daily);
po, per os (orally);
SE, standard error
Note:
Tumor volume (mm³) = (major axis [mm]) × (minor axis [mm]) × (minor axis [mm])/2
The control group was given the vehicle (0.5 w/v % HPMC solution).
* p < 0.05 with Aspin-Welch's t test as compared with the Control group.

TABLE 2
Effect of Compound A in immune deficient (CB1 7/Icr-Prkdc[scid]/CrlCrlj) mice

| | | | | Number | Tumor volume (mm³) | | Body weight change (%) |
|---|---|---|---|---|---|---|---|
| | Dose | Dosing | | of | Day 22 | | Day 22 |
| Group | (mg/kg/day) | Schedule | Route | Animals | Mean ± SE | | Mean ± SE |
| Control | 0 | qd, Day 1-21 | po | 5 | 1989.9 ± 151.5 | | 11.5 ± 2.2 |
| Compound A | 25.0 | qd, Day 1-21 | po | 5 | 2431.6 ± 359.7 | n.s. | 9.2 ± 4.2 |

Abbreviations:
qd, quaque die (once daily);
po, per os (orally);
SE, standard error;
n.s., not significant
Note:
Tumor volume (mm³) = (major axis [mm]) × (minor axis [mm]) × (minor axis [mm])/2
The control group was given the vehicle (0.5 w/v % HPMC solution).
n.s.: p > 0.05 with Aspin-Welch's t test as compared with the Control group.

TV of each group on Day 22 was analyzed by the Aspin-Welch's t-test, and the result showed that the Compound A administered group in wild type mice model exhibited an antitumor effect with significantly lower TV as compared to the control group. On the other hand, the Compound A administered group in immune deficient mice model didn't exhibit any antitumor effect as compared to the control group.

### Example 3: Effect of Combination of Biphenyl Compound and Immune Checkpoint Molecule Regulator in Colon Cancer Cell Transplanted Model

The MC38 cell suspension was transplanted subcutaneously at 2.0×10⁶ cells/0.1 ml to the right chest of 6-week-old C57BL/6JJcl mice (CLEA Japan, Inc.).

The tumor was grown until reaching a TV of 50 to 300 mm³ after the transplantation. Degimatic Caliper was used for tumor diameter measurement. The major diameter and the minor diameter of the tumor were measured, and TV was calculated from the following equation.

TV (mm³)=major diameter (mm)×minor diameter (mm)×minor diameter (mm)/2

By a stratified random allocation method using TV as an index, 10 mice were allocated to each group. The day when grouping was performed was defined as Day 0.

An electronic balance for animals was used for body weight measurement. The body weight change ratio on the nth day (BWCn) was calculated from the body weight on the nth day (BWn) in accordance with the following equation.

Body weight change ratio BWCn (%)=(BWn−BW0)/BW0×100

An appropriate amount of water for injection described in the Japanese Pharmacopeia was added for adjusting hypromellose concentration to 0.5 w/v %, and the mixture was then stirred with a stirrer to completely dissolve hypromellose, to prepare a 0.5% aqueous hypromellose solution. Benzoic acid salt of Compound A was ground with an agate mortar, and then suspended to a predetermined concentration with the 0.5% aqueous hypromellose solution, and ultrasonic treatment was performed to obtain a homogeneous suspension. The suspension containing Compound A was orally administered once a day for 21 consecutive days at a dose of 25 mg/kg/day. The dosage is indicated based on the free form of the Compound A.

The anti-mouse PD-1 antibody (anti-mPD-1 Ab) was prepared by diluting anti-PD-1, CD279 (PD-1) monoclonal antibody (Clone: RMP1-14, Bio X cell) to a predetermined concentration with Saline (Otsuka Pharmaceutical Factory, Inc.) immediately before administration. On the first day of administration (day 1), the diluted product was intraperitoneally administered with the dose of 0.05 mg/body of the anti-mouse PD-1 antibody.

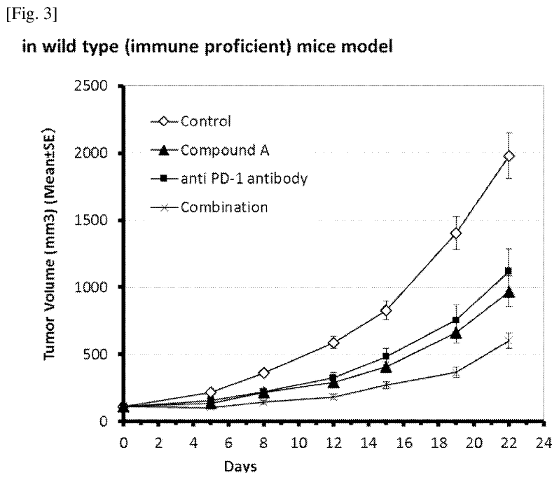

The results are shown in FIG. 3 and Table 3.

TABLE 3

| | | | | Number | Tumor Volume (mm³) | | Body weight change (%) |
|---|---|---|---|---|---|---|---|
| | | Dosing | | of | Day 22 | | Day 22 |
| Group | Dose | Schedule | Route | Animals | Mean ± SE | | Mean ± SE |
| Control | — | qd, Day 1-21 | po | 10 | 1980.4 ± 170.3 | | 5.8 ± 1.6 |
| Compound A | 25.0 mg/kg/day | qd, Day 1-21 | po | 10 | 969.3 ± 114.7 | * | 2.6 ± 1.2 |
| anti PD-1 antibody | 0.05 mg/body | Day 1 | ip | 10 | 1118.1 ± 167.9 | * | 13.5 ± 2.0 |
| Combination | Compound A_25.0 mg/kg/day | qd, Day 1-21 | po | 10 | 602.2 ± 56.7 | *#$ | 5.2 ± 2.1 |
| | anti PD-1 antbody_0.05 mg/body | Day 1 | ip | 10 | | | |

Abbreviations:
qd, quaque die (once daily);
po, per os (orally);
ip, intraperitoneal;
SE, standard error;
Note:
Tumor volume (mm³) = (major axis [mm]) × (minor axis [mm]) × (minor axis [mm])/2
The control group was given the vehicle (0.5 w/v % HPMC solution).
* p < 0.05 with Aspin-Welch's t test as compared with the Control group.
#: p < 0.05 with Aspin-Welch's t test as compared with the Compound A group.
$: p < 0.05 with Aspin-Welch's t test as compared with the anti PD-1 antibody group.

TV of each group on Day 22 was analyzed by the Aspin-Welch's t-test, and the result showed that both the single-agent group to which Compound A group or the antimouse PD-1 antibody was administered and the combination administration group to which Compound A plus anti-mouse PD-1 antibody were administered exhibited an antitumor effect with significantly lower TV as compared to the control group. Further, the combination administration group to which Compound A plus anti-mouse PD-1 antibody were administered had significantly lower TV and exhibited a higher antitumor effect as compared to the single-agent group to which Compound A or the anti-mouse PD-1 antibody was administered.

The average body weight change ratio of the combination administration group indicated no enhanced toxicity as compared to the single-agent group to which Compound A or the anti-mouse PD-1 antibody alone was administered.

1. An antitumor agent comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in a treatment of a subject having cancer, wherein the treatment comprises administering an immune checkpoint molecule regulator to the subject.

2. The antitumor agent according to claim 1, wherein the immune checkpoint molecule regulator is at least one or more selected from the group consisting of a PD-1 pathway antagonist and a CTLA-4 pathway antagonist.

3. The antitumor agent according to claim 1 or 2, wherein the immune checkpoint molecule regulator is a PD-1 pathway antagonist.

4. The antitumor agent according to claim 2 or 3, wherein the PD-1 pathway antagonist is at least one or more selected from the group consisting of an anti-PD-1 antibody, an anti-PD-L1 antibody and an anti-PD-L2 antibody.

5. The antitumor agent according to claim 2 or 3, wherein the PD-1 pathway antagonist is an anti-PD-1 antibody or an anti-PD-L1 antibody.

6. The antitumor agent according to claim 2 or 3, wherein the PD-1 pathway antagonist is an anti-PD-1 antibody.

7. The antitumor agent according to claim 2, wherein the CTLA-4 pathway antagonist is an anti-CTLA-4 antibody.

8. An antitumor effect enhancing agent for an immune checkpoint molecule regulator, comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof as an active ingredient.

9. An antitumor agent for treating a cancer patient given an immune checkpoint molecule regulator, the antitumor agent comprising 4-[5-[(3S)-3-aminopyrrolidine-1-carbonyl]-2-[2-fluoro-4-(2-hydroxy-2-methyl-propyl)phenyl] phenyl]-2-fluoro-benzonitrile or a salt thereof.

10. An antitumor agent for treating a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof, the antitumor agent comprising an immune checkpoint molecule regulator.

11. 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in treating a tumor by administration thereof in combination with an immune checkpoint molecule regulator.

12. 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in enhancing the antitumor effect of an immune checkpoint molecule regulator.

13. 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for use in treating a tumor in a cancer patient given an immune checkpoint molecule regulator.

14. An immune checkpoint molecule regulator for use in treating a tumor in a cancer patient given 4-[5-[(3S)-3-aminopyrrolidine-1-carbonyl]-2-[2-fluoro-4-(2-hydroxy-2-methyl-propyl)phenyl] phenyl]-2-fluoro-benzonitrile or a salt thereof.

15. A combination of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator for use in treating a tumor.

16. Use of 4-[5-[(3S)-3-aminopyrrolidine-1-carbonyl]-2-[2-fluoro-4-(2-hydroxy-2-methyl-propyl)phenyl] phenyl]-2-fluoro-benzonitrile or a salt thereof for producing an antitumor agent which is administered in combination with an immune checkpoint molecule regulator.

17. Use of 4-[5-[(3S)-3-aminopyrrolidine-1-carbonyl]-2-[2-fluoro-4-(2-hydroxy-2-methyl-propyl)phenyl] phenyl]-2-fluoro-benzonitrile or a salt thereof for producing an antitumor effect enhancing agent for enhancing the antitumor effect of an immune checkpoint molecule regulator.

18. Use of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof for producing an antitumor agent for treating a cancer patient given an immune checkpoint molecule regulator.

19. Use of an immune checkpoint molecule regulator for producing an antitumor agent for treating a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

20. Use of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator for producing an antitumor agent.

21. A method for treating a tumor, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator in combination.

22. A method for enhancing the antitumor effect of an immune checkpoint molecule regulator, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

23. A method for treating a tumor in a cancer patient given an immune checkpoint molecule regulator, comprising administering to a subject in need thereof 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof.

24. A method for treating a tumor in a cancer patient given 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof, comprising administering to a subject in need thereof an immune checkpoint molecule regulator.

25. A method for treating a tumor, comprising administering to a subject in need thereof a combination of 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator.

26. A pharmaceutical composition for preventing and/or treating a tumor, comprising 4-[5-[(3S)-3-aminopyrrolidine-1-carbonyl]-2-[2-fluoro-4-(2-hydroxy-2-methyl-propyl)phenyl]phenyl]-2-fluoro-benzonitrile or a salt thereof and an immune checkpoint molecule regulator.
